(19) United States
(12) Patent Application Publication
(10) Pub. No.: US 2024/0038354 A1
(43) Pub. Date: Feb. 1, 2024

(54) BIOMETRIC INFORMATION PROCESSING SYSTEM, BIOMETRIC INFORMATION PROCESSING DEVICE, BIOMETRIC INFORMATION PROCESSING METHOD, AND RECORDING MEDIUM

(71) Applicant: OMRON HEALTHCARE Co., Ltd., Kyoto (JP)

(72) Inventors: Hironori SATO, Kyoto (JP); Yoko KANEMITSU, Kyoto (JP)

(21) Appl. No.: 18/487,772

(22) Filed: Oct. 16, 2023

Related U.S. Application Data
(63) Continuation of application No. PCT/JP2022/019549, filed on May 6, 2022.

(30) Foreign Application Priority Data
May 25, 2021 (JP) 2021-087307

(51) Int. Cl. G16H 20/10; G16H 10/60

(52) U.S. Cl. CPC G16H 20/10; G16H 10/60

(57) ABSTRACT

A biometric information processing system including: measured value acquisition means that acquires a measured value related to biometric information of a patient; comparison means that compares the measured value with an improvement target reference set in advance; dosing information acquisition means that acquires dosing information including at least information on a dosing frequency of the patient when the measured value has not achieved the improvement target reference; medicinal effect confirmation support image generation means that generates a medicinal effect confirmation support image in which measured value transition information indicating a change in the measured value of the patient at each first predetermined period and the dosing information are browsable when the measured value has not achieved the improvement target reference; warning information generation means that generates warning information when the measured value has not achieved the improvement target reference.

### CROSS-REFERENCE TO RELATED APPLICATIONS

This application is the U.S. national stage application filed pursuant to 35 U.S.C. 365(c) and 120 as a continuation of International Patent Application No. PCT/JP2022/019549, filed May 6, 2022, which application claims priority to Japanese Patent Application No 2021-087307, filed May 25, 2021, which applications are incorporated herein by reference in their entireties.

### TECHNICAL FIELD

The present invention belongs to a technical field related to healthcare, and relates to a biometric information processing system, a biometric information processing device, a biometric information processing method, and a program.

### BACKGROUND ART

In recent years, there has been an increasing need for remote medical care, and as a part of that, there has been proposed a monitoring system in which biometric information, such as a blood glucose level or a blood pressure value measured by a patient at home, is transmitted to a database, a medical professional gives a warning that the biometric information is out of a range of a target value set in advance, the medical professional checks the state and gives instructions through a video call while viewing the measurement results, or a doctor considers changing a medication (see Patent Literature 1).

However, in such a conventional system, regardless of the relationship between the state of the measured value of the biometric information and the target value, there is a problem that the workload of the medical professional increases as the amount of data to be examined by the medical professional increases.

Moreover, since the deviation between the expected (assumed) change (improvement effect) and the measured value, which differs depending on the treatment strategy, is not evaluated, there is a problem that it is unclear whether or not the currently adopted treatment strategy is appropriate and should be continued in the future.

In addition, even in the conventional system, the degree of compliance with an approach (hereinafter, also referred to as a target action) to be performed by the patient for treatment, such as medication, is monitored, but the relationship between the degree of compliance and the therapeutic effect is not evaluated. Therefore, there is a problem that, when an improvement effect is not observed (or is not achieved as expected), it cannot be distinguished whether the treatment strategy is inappropriate or the degree of compliance with the target action is low.

Furthermore, in the monitoring of the degree of compliance with the target action to be performed by the patient, input to the system is required for each daily life event, such as meal, medication, exercise, and measurement of biometric information, on the patient side, and the burden on the patient is extremely large. For this reason, the patient does not always fully cooperate with the monitoring, and there is a possibility that accurate monitoring cannot be performed.

### CITATION LIST

### Patent Literature

Patent Literature 1: US 2017/235909 A

### SUMMARY OF INVENTION

### Technical Problem

In view of the above-described problems, an object of the present invention is to provide a technique capable of reducing the workload of a medical professional and a patient in a system related to remote medical care.

### Solution to Problem

The present invention adopts the following configurations to solve the above-described problems. That is, a biometric information processing system includes: measured value acquisition means that acquires a measured value related to biometric information of a patient;
comparison means that compares the measured value with an improvement target reference set in advance;
dosing information acquisition means that acquires dosing information including at least information on a dosing frequency of the patient when the measured value has not achieved the improvement target reference;
medicinal effect confirmation support image generation means that generates a medicinal effect confirmation support image in which measured value transition information indicating a change in the measured value of the patient at each first predetermined period and the dosing information are browsable when the measured value has not achieved the improvement target reference;
warning information generation means that generates warning information thereon when the measured value has not achieved the improvement target reference;
first output means that outputs the warning information when the measured value has not achieved the improvement target reference; and
second output means that outputs the medicinal effect confirmation support image when the measured value has not achieved the improvement target reference.

Here, the “first predetermined period” is not particularly limited and can be set to any period. For example, it may be one week, one month, one day, or several hours. In addition, the “improvement target reference” may be a general-purpose so-called normal value or the like, or may be a value decided each time according to the patient's state, a prescription, or the like. Also, the “output means” may be a display device, such as a liquid crystal display, or may be a printing device, such as a printer. Note that the first output means and the second output means may be the same output means. That is, the generated warning information may be displayed on a display device or the like that outputs the medicinal effect confirmation support image, or may be output as an image or sound via a portable device, such as a mobile phone, that is separately used by a medical professional. The warning information is not limited to what is called an alarm that is output immediately. For example, in a system related to patient management, such as an electronic medical chart, which is regularly checked by a doctor, information (mark display, warning sound, or the like) indicating that the subject is a warning target may be output at the time of the check.

With such a configuration, when the measured value of the biometric information has not reached the improvement target reference set in advance, warning information thereon is generated, and the warning can be output via any output means. Therefore, when there is a patient whose measured value of the biometric information related to the measurement has not reached the target, a medical professional can easily notice the fact. In addition, since the measurement data of the patient can be output as an image (medicinal effect confirmation support image) including the data indicating the transition of the measurement data at each predetermined period and the information on the dosing frequency of the patient in a browsable manner, the medical professional (especially a doctor) can check the image and promptly decide whether or not the current treatment strategy is appropriate. Also, when the patient complies with a target action and the measured value achieves the improvement target reference, it is not necessary to perform special troublesome input to the system or the like, and thus the burden required on the patient side can be reduced as much as possible.

Further, the comparison means may compare an average value of the measured values in the first predetermined period with the improvement target reference, and the dosing information acquisition means may acquire dosing information including information on a dosing frequency of the patient in the first predetermined period when the average value has not achieved the improvement target reference.

When the biometric information is measured every day for a predetermined period (for example, one week), the measured values of the biometric information within the predetermined period would vary naturally. However, by treating the average value of these measured values as one measurement data in the predetermined period, the comparison with the improvement target reference can be appropriately performed.

Further, the biometric information may be information related to blood pressure,
the measured value acquisition means may acquire a systolic blood pressure value and a diastolic blood pressure value as the measured values for each measurement,
the improvement target reference to be compared by the comparison means may be set for each of the systolic blood pressure value and the diastolic blood pressure value,
the dosing information acquisition means may acquire the dosing information when either of the systolic blood pressure value or the diastolic blood pressure value has not achieved the improvement target reference set for each of the systolic blood pressure value and the diastolic blood pressure value, and
the warning information generation means may generate the warning information when either of the systolic blood pressure value or the diastolic blood pressure value has not achieved the improvement target reference set for each of the systolic blood pressure value and the diastolic blood pressure value.

When the biometric information to be measured is blood pressure (for example, in a case of a patient with hypertension), it is necessary to control both blood pressure values of systolic blood pressure and diastolic blood pressure. Therefore, the above-described configuration is preferable.

Further, the measured value acquisition means may further acquire time period information for specifying a time period at which the blood pressure has been measured for each measurement; and
the measured value transition information may indicate changes in the systolic blood pressure value and the diastolic blood pressure value at each of the first predetermined periods for each of the time periods.

Here, the time period information may be, for example, time data indicating date and time. In the case of measuring blood pressure, it is recommended to perform the measurement twice, after getting up (before breakfast) and before going to bed. Therefore, the above-described configuration in which measurement data can be managed for each of the time periods is preferable.

Further, the biometric information processing system may further include a patient-side information processing terminal that executes, when the measured value has not achieved the improvement target reference, an automatic medical interview processing of requesting the patient to input at least information related to a dosing frequency of the patient, and
the dosing information acquisition means may acquire the dosing information input from the patient through automatic medical interview processing executed in the patient-side information processing terminal.

With such a configuration, for example, as a function of an application of a portable information processing terminal, such as a smartphone used by a patient, it is possible to acquire subjective information from the patient himself/herself through a question and answer with a so-called chatbot. Note that the means for acquiring the dosing information may be any means, and is not limited to such an aspect. For example, a sensor that detects removal of a medicine may be provided in a container in which the patient stores the medicine, and the dosing information may be acquired from information (the amount of the medicine removed, and the date and time of the removal) acquired by the sensor. Also, the patient may be linked with a database of a pharmacy from which the medicine is purchased, and the dosing information may be acquired from the sales history.

Further, the dosing information may include information on a presence or absence and a type of a side effect caused in the patient by a medication, and the automatic medical interview processing may further request input of information on the presence or absence and the type of the side effect caused in the patient by the medication. One reason why a patient does not take a medicine according to the dosage regimen is the presence of side effects. Therefore, by additionally acquiring information related to side effects according to the configuration as described above, the information can be used to consider an appropriate treatment strategy.

Further, the biometric information processing system may further include action evaluation information generation means that generates, based on the measured value and the dosing information in a second predetermined period, action evaluation information including information related to evaluation of an approach for treatment of the patient in the second predetermined period, and the patient-side information processing terminal to which the action evaluation information is output. Note that the “second predetermined period” referred to here may be the same period as the first predetermined period or may be a different period. With such a configuration, a patient can receive feedback according to his/her approach for treatment, and therefore it is possible to support maintenance of motivation to continue the treatment.

Further, the biometric information processing system may further include recommended treatment information generation means that generates, when the measured value has not achieved the improvement target reference, recommended treatment information related to a treatment strategy recommended to the patient, and the recommended treatment information may be further output to the second output means. With such a configuration, the doctor can consider the treatment strategy while referring to the recommended treatment information, and decide on (change) the treatment strategy more promptly.

Further, the medicinal effect confirmation support image may include information on a difference value between the measured value and the improvement target reference. This allows the control state of the biometric information to be more easily understood, and the medical professional to make a more prompt determination.

Further, the biometric information processing system may further include annotation information generation means that generates annotation information related to a matter to be noted by a medical professional in charge of the patient based on the measured value, and the annotation information may be further output to the second output means. In addition, when there is such annotation information, warning information thereon may be output.

For example, in a case where the measured value of blood pressure has achieved the improvement target reference but is in a state of hypotension for reasons such as the effect of a medicine being too strong, the measured value of blood pressure is apparently under control and appears to have no problem. In such a case, by marking annotation information for calling attention to, for example, the fact of being in a hypotensive state, or alerting the presence of such information, the medical professional can be aware of the state that requires attention (or handling) at an early stage. Note that the content of the annotation information is not particularly limited, and may be, for example, information suggesting the possibility of secondary hypertension based on various data.

Further, the improvement target reference may be a value of the biometric information after a change that is assumed when the patient takes a medicine prescribed for the patient for the first predetermined period in accordance with a usage and a dose. This allows the target value for each predetermined period to be decided in accordance with the current state, which helps maintain motivation of the patient. In addition, as compared with the case where a so-called normal value or the like is used as the improvement target reference, there is a high possibility that the failure to achieve the reference suggests that the treatment strategy (such as the adopted medicine) is not appropriate. Therefore, it is possible to review the treatment strategy at an early stage.

In addition, the present invention can also be considered as a biometric information processing device including the measured value acquisition means, the comparison means, the dosing information acquisition means, the medicinal effect confirmation support image generation means, and the warning information generation means, and constituting at least a part of the biometric information processing system.

In addition, the present invention can also be considered as a program for

causing a computer to function as such a biometric information processing device, and a computer-readable recording medium including such a program recorded therein in a non-transitory manner.

In addition, the present invention can also be considered as a biometric information processing method including:
acquiring a measured value related to biometric information of a patient;
comparing the measured value with an improvement target reference set in advance;
acquiring dosing information including at least information on a dosing frequency of the patient when the measured value has not achieved the improvement target reference;
generating a medicinal effect confirmation support image in which measured value transition information indicating a change in the measured value of the patient at each first predetermined period and the dosing information are browsable when the measured value has not achieved the improvement target reference;
generating warning information when the measured value has not achieved the improvement target reference;
outputting the warning information when the measured value has not achieved the improvement target reference; and
outputting the medicinal effect confirmation support image when the measured value has not achieved the improvement target reference.

Also, the configurations and processing described above can be combined with one another to constitute the present invention unless the combination leads to contradiction.

### Advantageous Effects of Invention

With the present invention, there can be provided a technique in a system related to remote medical care that allows deciding on whether or not a current treatment method is appropriate at an early stage, and reducing the workload of a medical professional and a patient.

### BRIEF DESCRIPTION OF DRAWINGS

Various embodiments are disclosed, by way of example only, with reference to the accompanying schematic drawings in which corresponding reference symbols indicate corresponding parts, in which:

FIG. 1 is a schematic diagram illustrating a configuration of a biometric information processing system 1 according to an embodiment.

FIG. 2 is a flowchart schematically illustrating a flow of remote medical care performed in the embodiment.

FIG. 3 is a block diagram illustrating a functional configuration of a server device according to the embodiment.

FIG. 4 is a block diagram illustrating a functional configuration of a doctor-side terminal according to the embodiment.

FIG. 5 is a first diagram illustrating a screen example output on the doctor-side terminal.

FIG. 6 is a second diagram illustrating a screen example output on the doctor-side terminal.

FIG. 7 is a third diagram illustrating a screen example output on the doctor-side terminal.

FIG. 8 is a block diagram illustrating a functional configuration of a patient-side terminal according to the embodiment.

FIG. 9(A) is a first diagram illustrating a screen example at the time of executing an automatic medical interview program according to the embodiment. FIG. 9(B) is a second diagram illustrating a screen example at the time of executing the automatic medical interview program according to the embodiment.

FIG. 10 is a diagram illustrating a flow of information exchange and processing performed in the biometric information processing system according to the embodiment.

FIG. 11 is a diagram illustrating an outline of a biometric information processing system 2 according to a modified example of the embodiment.

### DESCRIPTION OF EMBODIMENTS

Embodiment 1

Embodiments of the present invention will be specifically described below

with reference to the drawings. However, it should be noted that the dimension, shape, relative arrangement and the like of the components described in this embodiment are not intended to limit the scope of this invention to them alone, unless otherwise stated.

System Configuration

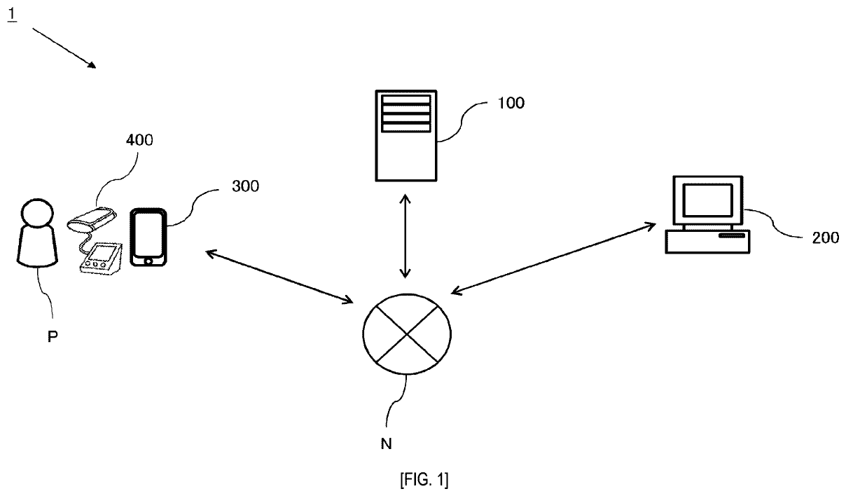

FIG. 1 is a schematic diagram illustrating a configuration of a biometric information processing system 1 according to the present embodiment. As illustrated in FIG. 1, the biometric information processing system 1 is constituted including a server device 100, a doctor-side terminal 200 used by a doctor, and a patient-side terminal 300 and a blood pressure monitor 400 used by a patient P. These respective configurations can communicate with one another via a communication network N.

The biometric information processing system 1 according to the present embodiment is a system related to remote medical care, and transmits a blood pressure value measured by a patient at home to the server device 100 via the network N, processes the information, and provides the information to a medical professional, thereby assisting a doctor in performing treatment on the patient.

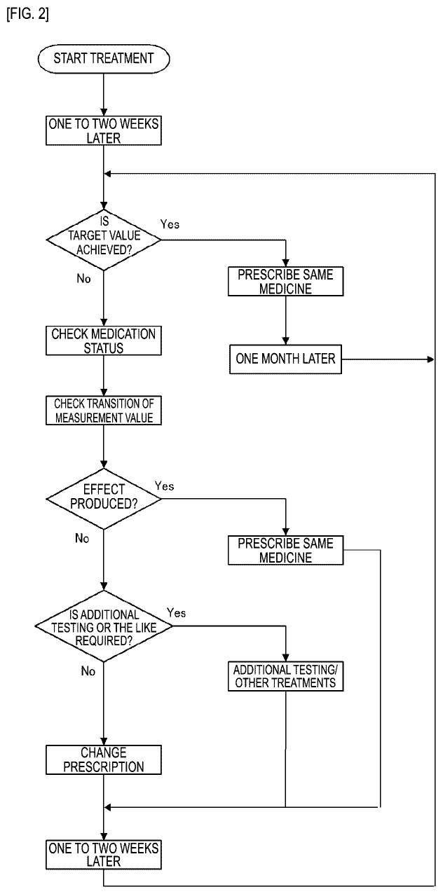

FIG. 2 is a flow chart schematically illustrating a flow of the remote medical care performed in the present embodiment. Hereinafter, the remote medical care performed in the present embodiment will be specifically described with reference to FIG. 2. That is, a patient who has received a definite diagnosis of hypertension starts medication treatment in accordance with a doctor's prescription, and continuously measures his/her blood pressure at home. The biometric information processing system 1 collects the measured values, and after a predetermined period of time has elapsed (here, one to two weeks later), it is determined whether or not a target value set in advance is achieved (hereinafter, the fact that the measured value achieves the target value is also expressed as “being controlled” or the like). Here, the measured value achieving the target value means, for example, that the blood pressure value is less than a predetermined threshold. When the measured value is in a controlled state, the same medicine continues to be taken, and after a predetermined period of time (here, one month) has elapsed, it is determined whether or not the measured value of blood pressure is in a controlled state.

On the other hand, when the target value is not achieved, the medication state is checked to see whether the patient has been taking the medicine according to the prescription. In addition, the transition of the measured value at each predetermined period is checked by the doctor, and is used for deciding on a treatment strategy together with the information on the medication state.

When the doctor determines from the state of transition of the measured value that a certain effect is produced by the medication, the doctor decides to continuously administer the same medicine (wait and see). In that case, it is determined again after a predetermined period (here, one to two weeks) has elapsed, whether or not the measured value of blood pressure is in a controlled state.

Also, when the medication does not seem to be effective, the doctor considers the possibility of other diseases and decides to perform additional testing or treatment (including prescription) taking into account the other diseases. In that case, it is determined again after a predetermined period (here, one to two weeks) has elapsed, whether or not the measured value of blood pressure is in a controlled state.

In addition, when other diseases are not the causes and the state is expected to improve by changing the medicine being taken, such as stopping the medication due to a side effect, the prescription contents are changed. In that case, it is determined again after a predetermined period (here, one to two weeks) has elapsed, whether or not the measured value of blood pressure is in a controlled state.

By repeatedly performing the flow described above, the remote medical care of the hypertensive patient is executed. The biometric information processing system 1 according to the present embodiment supports the execution of such a flow, and reduces the burden on both the doctor (medical professional) and the patient. Hereinafter, the respective configurations of the system will be described in detail.

Server Device

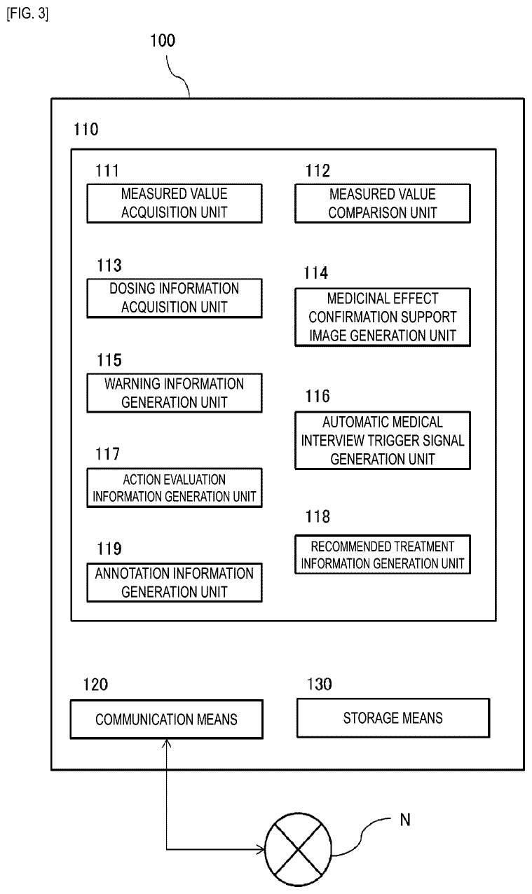

FIG. 3 is a block diagram illustrating a functional configuration of the server device 100. The server device 100 is constituted of a general server computer and, as illustrated in FIG. 3, includes a control unit 110, communication means 120, and storage means 130.

The control unit 110 is means that manages the control of the server device 100, and is constituted of a processor, such as a Central Processing Unit (CPU) or a Digital Signal Processor (DSP). The control unit 110 includes, as functional modules related to biometric information management, respective functional units of a 30 measured value acquisition unit 111, a measured value comparison unit 112, a dosing information acquisition unit 113, a medicinal effect confirmation support image generation unit 114, a warning information generation unit 115, an automatic medical interview trigger signal generation unit 116, an action evaluation information generation unit 117, a recommended treatment information generation unit 118, and an annotation information generation unit 119. These respective functional units will be described in detail below.

The communication means 120 is communication means for connecting the server device 100 to the communication network N, and is constituted including, for example, a communication interface board and a wireless communication circuit for wireless communication.

Although not illustrated, the storage means 130 includes a main storage unit, such as a Read only memory (ROM) or a Random access memory (RAM), and an auxiliary storage unit, such as an EPROM, a Hard Disk Drive (HDD), a Solid State Device (SSD), or a removable medium. The auxiliary storage unit stores an operating system (OS), various programs, and the like. By the stored programs being loaded into a work area of the main storage unit and executed, and the respective component units and the like being controlled by the execution of the programs, the respective functional units that meet predetermined purposes can be realized.

The measured value acquisition unit 111 acquires a measured value measured by the patient P with the blood pressure monitor 400 via the communication network N and stores the measured value in the storage means 130. The measured value comparison unit 112 compares the acquired measured value (specifically, an average value of measured values for each predetermined period, such as one week) with a target value set in advance for each individual patient P. Thus, it is determined whether or not the blood pressure value of the patient P is controlled. The target value may be stored in advance in the storage means 130, for example.

When the blood pressure value of the patient P is not controlled (that is, the target is not achieved), the automatic medical interview trigger signal generation unit 116 generates a signal for executing an automatic medical interview in the patient-side terminal 300 as described below. The generated signal is transmitted from the communication means 120 and received by the patient-side terminal 300. In addition, the dosing information acquisition unit 113 acquires dosing information including information of a dosing frequency of the patient P by the automatic medical interview executed by the patient-side terminal 300.

When the blood pressure value of the patient P is not controlled, the warning information generation unit 115 further generates warning information to be output to the doctor-side terminal 200 as described below. The generated warning information is transmitted from the communication means 120 and received by the doctor-side terminal 200.

The annotation information generation unit 119 generates annotation information related to a matter to be noted by the doctor in charge of the patient P based on the acquired measured value. Specifically, for example, in a case where the blood pressure appears to be controlled, but actually the medicine is too effective and is causing a hypotensive state, the annotation information thereon is generated.

The medicinal effect confirmation support image generation unit 114 generates a medicinal effect confirmation support image in which measured value transition information indicating a change in the acquired measured values at each predetermined period and dosing information are browsable. In addition, the annotation information may also be displayed together on the medicinal effect confirmation support image. The generated medicinal effect confirmation support image is transmitted to the doctor-side terminal 200 via the communication network N.

The action evaluation information generation unit 117 generates action evaluation information including information related to evaluation of a target action of the patient P in a predetermined period based on the measured values and the dosing information in the predetermined period. Here, the information related to the evaluation of the target action may be, for example, a graph of the transition of the measured value. Also, it can be a message corresponding to the control state of the measured value (for example, when the target is achieved, a phrase praising the fact and the effort, and encouraging a continuous effort), as an example. The generated action evaluation information is transmitted from the communication means 120 at an appropriate timing and received by the patient-side terminal 300.

In a case where there is a treatment strategy (prescription) recommended for improving the medical condition of the patient P based on the measured values and the dosing information (and other information), the recommended treatment information generation unit 118 generates information for suggesting the treatment strategy to the doctor. The generated information may be included in the medicinal effect confirmation support image, or may be separately outputtable on the doctor-side terminal 200.

Doctor-Side Terminal

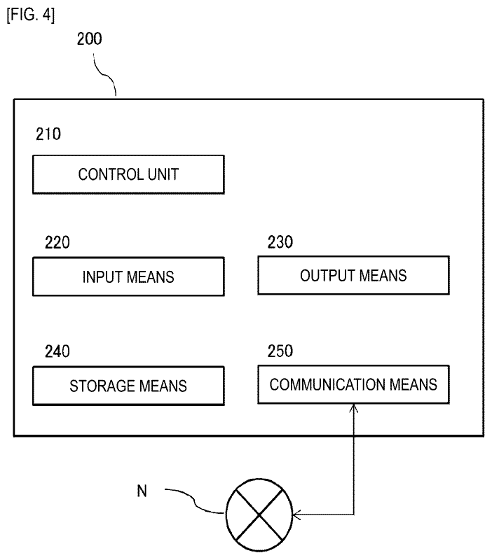

FIG. 4 is a block diagram illustrating a functional configuration the doctor-side terminal 200. The doctor-side terminal 200 is a general computer, such as a fixed personal computer, a portable notebook-type personal computer, or a tablet device, and includes a control unit 210, input means 220, output means 230, storage means 240, and communication means 250.

The control unit 210 is means for controlling the doctor-side terminal 200, and is constituted of, for example, a CPU. The input means 220 is means that receives information input from the outside, such as a keyboard, a mouse, a touch panel, a camera, or a microphone. The output means 230 is constituted including a liquid crystal display, a speaker, and the like. The storage means 240 is constituted similarly to the server device including a main storage unit, an auxiliary storage unit, and the like, and stores various data acquired via the communication network N, such as an operating system (OS) and various programs. The communication means 250 is constituted including, for example, a communication interface board and a wireless communication circuit for wireless communication.

Note that, although not illustrated, the doctor-side terminal may be accessible to an electronic medical chart management system. In such a case, electronic medical chart data of a patient stored in the electronic medical chart management system may be read out to be transmitted to the server device, and warning information and the like transmitted from the server device 100 may be associated with the electronic medical chart data. In such a case, the doctor can also check the warning information via the electronic medical chart management system.

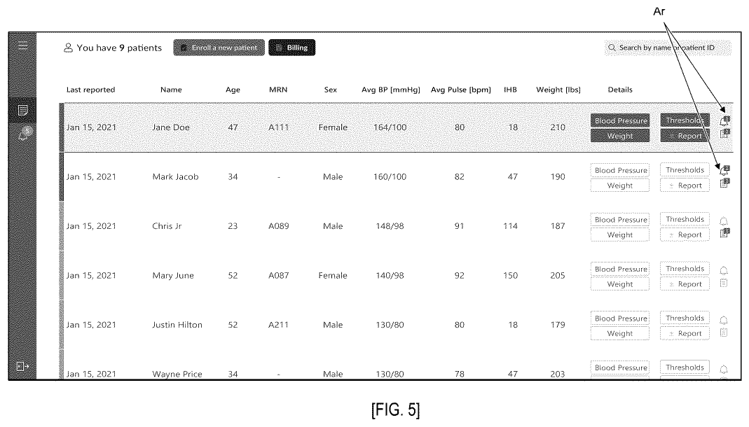

In the doctor-side terminal 200, the warning information and the medicinal effect confirmation support image are acquired from the server device 100 via the communication network N, and these pieces of information are output to the output means 230. FIG. 5 to FIG. 7 illustrate examples of screens displayed on the output means 230 of the doctor-side terminal 200. FIG. 5 is a screen displaying a list of patients assigned to a doctor as a manager of the terminal, and for patients whose measured value has not achieved the target, warning information Ar (a bell mark and a numeral notation) thereon is also displayed.

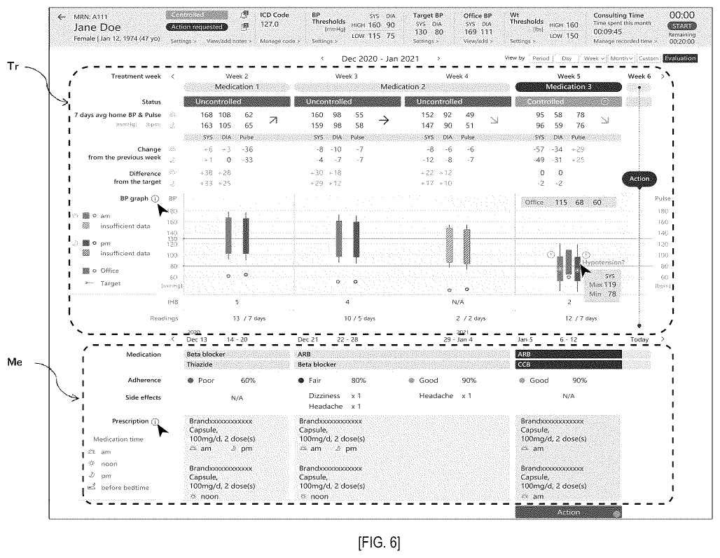

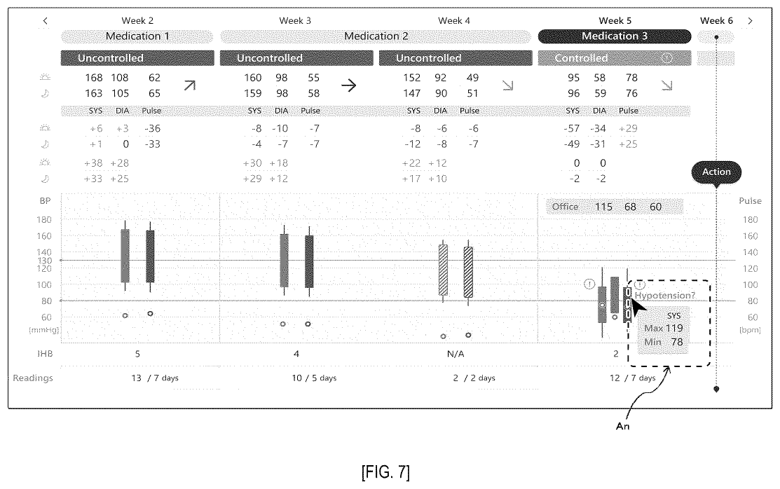

FIG. 6 is a management screen including a medicinal effect confirmation support image of an individual patient P, and is a screen including measured value transition information Tr and dosing information Me. FIG. 7 is a partially closed-up screen of the measured value transition information Tr in the medicinal effect confirmation support image of FIG. 6. In the present embodiment, annotation information An is also displayed in the portion of the measured value transition information Tr. The annotation information An in FIG. 7 is information suggesting the risk of hypotension.

As illustrated in FIG. 6 and FIG. 7, in the measured value transition information Tr in the present embodiment, the average values (7 days avg home BP & Pulse) of the systolic blood pressure (SYS), the diastolic blood pressure (DIA), and heart rate in one week are displayed with one week as a predetermined period (a period as one group). More specifically, one week average values of each SYS and DIA are displayed for each of the time periods in the morning (after getting up) and at night (before going to bed). These values are not only depicted by numerical values, but also depicted as a graph in which a range between SYS and DIA is filled in a bar shape and a relationship with a threshold line indicating a target value is visually indicated. The lines extending upward and downward from the bar-shaped portions of the graph indicate the variation of the measured values in the measurement for one week, and the line extending upward indicates the systolic blood pressure value of the week and the line extending downward indicates the diastolic blood pressure value. In addition, the week in which the bar-shaped portion is hatched indicates that the information is not reliable because the amount of the measurement data is small.

In addition, in the measured value transition information Tr, a change amount (Change from the previous week) in a case of comparing with a numerical value of the previous week and a value (Difference from the target) indicating a deviation from the target value (in this example, SYS is 130 mmHg and DIA is 80 mmHg) are also indicated.

In addition, in the dosing information Me of the present embodiment, information such as the type of medicine taken by the patient P in that week, the frequency of dosing (in this example, indicated as Adherence by a dose rate), and a symptom and frequency of a complained side effect are described. In addition, information (Prescription) indicating prescription contents, such as a brand, a dosage form, and a dose amount of a medicine, can also be referred to.

By referring to such a screen that allows browsing the measured value transition information Tr and the dosing information Me, the doctor can easily and promptly determine what kind of treatment strategy should be adopted for the patient P.

Patient-Side Terminal

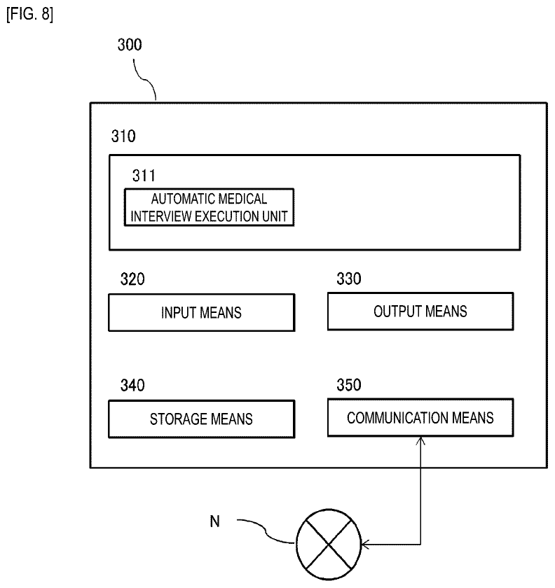

FIG. 8 is a block diagram illustrating a functional configuration of the patient-side terminal 300. The patient-side terminal 300 is, for example, a portable information processing terminal, such as a smartphone, a tablet terminal, or a wristwatch type wearable terminal, and includes a control unit 310, input means 320, output means 330, storage means 340, and communication means 350.

The control unit 310 is means for controlling the patient-side terminal 300, and

is constituted of, for example, a CPU. As the input means 320, a touch panel display integrated with the output means 330 or the like can be adopted. Similarly to other terminals, the storage means 340 is constituted including a main storage unit, an auxiliary storage unit, and the like, and stores various data acquired via the communication network N, such as an operating system (OS) and various programs. The communication means 250 is constituted including, for example, a wireless communication circuit for wireless communication.

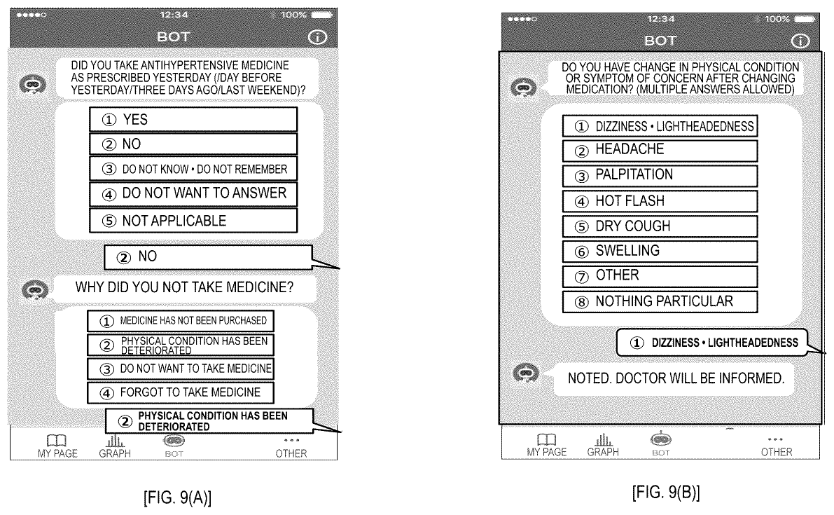

The control unit 310 includes an automatic medical interview execution unit 311 as a functional module related to biometric information management. Upon receiving the automatic medical interview trigger signal transmitted from the server device 100, the automatic medical interview execution unit 311 executes an application for automatically performing a medical interview about the frequency of dosing, the presence or absence of a side effect, and the type thereof. The automatic medical interview application can adopt a form such as a chatbot, for example, and may be stored in advance in the storage means 340 of the patient-side terminal 300 or may be provided in the form of an Application Service Provider (ASP) in the server device 100. FIG. 9(A) and FIG. 9(B) illustrate a screen example of when the automatic medical interview program is executed. FIG. 9(A) illustrates a medical interview screen relating to medication, and FIG. 9(B) illustrates a medical interview screen relating to side effects.

The answer made by the patient P to the automatic medical interview as described above is transmitted from the communication means 350 to the server device 100 via the network N. In addition to measurement data acquired from the blood pressure monitor 400 described below, necessary information input by the patient P is transmitted to the server device 100.

In addition, the patient-side terminal 300 receives the action evaluation information including the information related to the evaluation of the approach from the server device 100 according to the result of the approach for the treatment of the patient P, and the content is output to the output means 330.

Blood Pressure Monitor

The blood pressure monitor 400 is used by the patient P for daily blood pressure measurement, and may have any kind of form. For example, it may be a general stationary type, a portable type in which a portable cuff is wrapped around an upper arm or the like, or may be of a wearable type to be worn on a wrist of a patient. The blood pressure is measured by the oscillometric method by an operation of the patient P (or, in the case of a wearable type, at a timing or time interval set in advance), and the blood pressure data is wirelessly transmitted to the patient-side terminal 300 by wireless communication, for example. As a communication interface used between the blood pressure monitor 400 and the patient-side terminal 300, a short-range wireless data communication standard, such as Bluetooth (trade name) or infrared communication, can be adopted.

The blood pressure monitor 400 need not have communication means. In that case, the patient P may manually input measurement data (including a blood pressure value and a measurement date and time) to the patient-side terminal 300, and the information may be transmitted to the server device 100. Note that the measurement data may include heart rate.

Further, the patient-side terminal 300 may also have the function of the blood pressure monitor 400. For example, in a case where the patient-side terminal 300 is a wearable terminal worn on the wrist of the patient P, when a blood pressure measurement function is provided in the wearable terminal, the wearable terminal can also serve as the blood pressure monitor 400. Conversely, for example, the stationary blood pressure monitor 400 may have a function as an information processing terminal, and also serve as the patient-side terminal 300.

The measurement method of the blood pressure monitor 400 may be a method in which measurement is performed for each heartbeat, or a trigger measurement method in which blood pressure fluctuation is estimated from pulse transit time and blood pressure is measured in a spot manner using the fluctuation as a trigger.

Flow of Information Processing in System

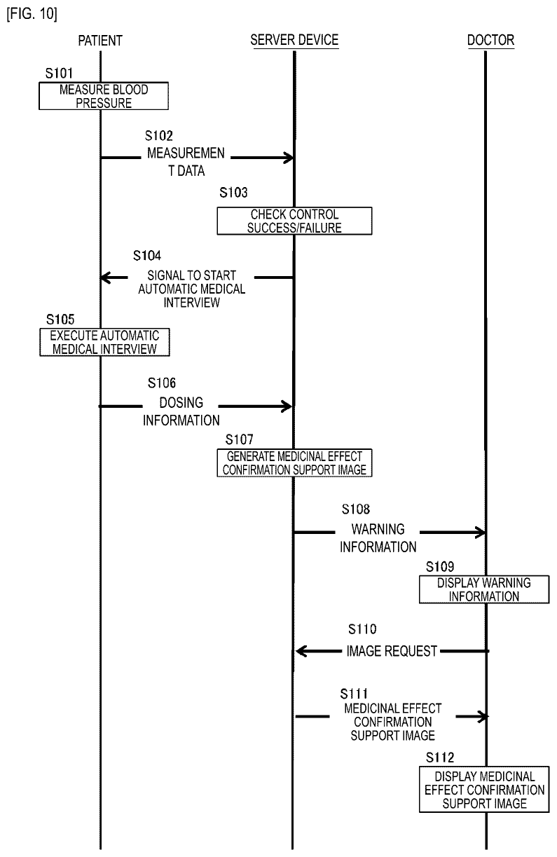

Next, a flow of information processing performed in the biometric information processing system 1 according to the present embodiment having the above-described configuration will be described. FIG. 10 is a diagram indicating the flow of information exchange and processing performed in the biometric information processing system 1. As illustrated in FIG. 10, first, the patient P measures his/her blood pressure with the blood pressure monitor 400 (S101). The measurement data is transmitted to the server device 100 via the patient-side terminal 300 each time or collectively for a predetermined period (for example, one week) (S102).

In the server device 100, the received measurement data is stored in the storage

means 130, and based on the information, it is checked whether the blood pressure of the patient P is controlled by comparing the data with predetermined target values (S103). Here, different target values are set for the systolic blood pressure and the diastolic blood pressure, and it is determined whether or not the average values of the systolic blood pressure and the diastolic blood pressure for each predetermined period achieve the respectively corresponding target values. When the blood pressure is not controlled (that is, either the systolic blood pressure or the diastolic blood pressure exceeds the target value), a trigger signal is transmitted to the patient-side terminal 300 to start an automatic medical interview for inquiring about the dosing frequency and the like (S104).

In the patient-side terminal 300 that has received the trigger signal of the automatic medical interview, the automatic medical interview program is executed, and the patient P inputs the dosing information including the dosing frequency (S105). Then, the dosing information is transmitted to the server device 100 (S106). The server device 100 generates a medicinal effect confirmation support image including information related to the transition of the blood pressure value and information related to the dosing frequency of the patient P (S107), and transmits warning information to the effect that the blood pressure of the patient P is in an uncontrolled state to the doctor-side terminal 200.

When the doctor-side terminal 200 receives the warning information, the warning information is output (S109). Specifically, on the display screen as the output means 230, the mark as illustrated in FIG. 5 may be displayed as described above, another alert screen may be displayed, or output by voice may be performed. Note that the warning information may be output by another device, such as a cellular phone used by a doctor, separately from the doctor-side terminal 200.

In order for the doctor to refer to the medicinal effect confirmation support image of the patient P to which the warning has been issued, request information for displaying the medicinal effect confirmation support image is transmitted from the doctor-side terminal 200 to the server device 100 (S110). Then, the server device 100 that has received the request provides the medicinal effect confirmation support image to the doctor-side terminal 200 (S111), and the medicinal effect confirmation support image is displayed on the output means 230 of the doctor-side terminal 200 (S112). Here, data of the medicinal effect confirmation support image may be transmitted to the doctor-side terminal 200 and stored in the storage means 240 of the doctor-side terminal 200, or may be provided in the form of an ASP and image data may be unable to be stored. The content of the medicinal effect confirmation support image is as described above.

With the biometric information processing system 1 according to the present embodiment as described above, when there is a patient whose blood pressure is not controlled well, the doctor can promptly recognize the fact by receiving the warning information, and can efficiently decide on (change) the treatment strategy of the patient by examining the medicinal effect confirmation support image provided. Specifically, since chronological data of blood pressure is displayed for each determined period and information on the medication of the patient in the predetermined period can be referred to instantly, even for a poorly controlled person, it can be easily determined whether a treatment is starting to produce effect, whether a prescription should be changed, whether other diseases should be suspected, or the like.

Also, besides the blood pressure measurement, the patient does not need to perform any troublesome input operation other than responding to the automatic medical interview when the blood pressure is not controlled, and therefore the complexity of using the system can be reduced as much as possible. Moreover, since the action evaluation information is appropriately distributed with respect to the target action, such as medication, it is possible to support the maintenance of motivation to continue the treatment.

Modified Example

The description of the embodiments described above is merely illustrative of

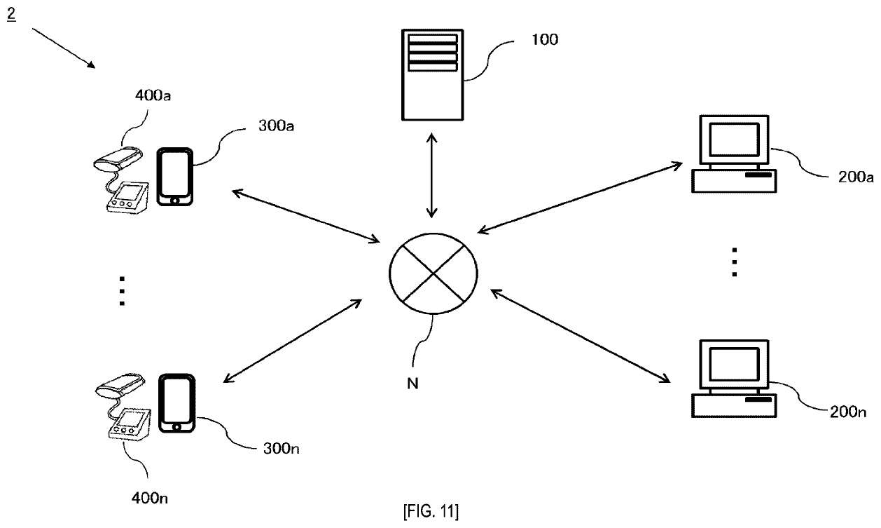

the present invention, and the present invention is not limited to the specific embodiments described above. Within the scope of the technical idea of the present invention, various modifications and combinations can be made. For example, in the above-described embodiment, a configuration including one of each the doctor-side terminal 200 and the patient-side terminal 300 has been described. However, as illustrated in FIG. 11, the present invention can be applied to a biometric information processing system 2 including a plurality of doctor-side terminals 200a to 200n and/or a plurality of patient-side terminals 300a to 300n.

Also, the means for acquiring the dosing information is not limited to the

automatic medical interview program by the chatbot. For example, a sensor that detects removal of a medicine may be provided in a container of the medicine to be taken by the patient P, and the dosing information may be acquired based on information (dosing frequency, dosing amount, and the like) related to the removal of the medicine detected by the sensor. In addition, by associating with a system of a pharmacy used by the patient P, the dosing information may be acquired from information of a sold medicine, information of a sales period, or the like.

Further, in the above-described embodiment, as an example of remote treatment of a hypertensive patient, a case where the biometric information to be processed is blood pressure value (and heart rate) has been described. However, it is needless to say that the present invention can be applied to other diseases and biometric information, such as blood glucose level of a diabetic patient.

### REFERENCE SIGNS LIST

1, 2 . . . Biometric information processing system
100 . . . Server device
110, 210, 310 . . . Control unit
120, 240, 340 . . . Storage means
130, 250, 350 . . . Communication means
200 . . . Doctor-side terminal
220, 320 . . . Input means
230, 330 . . . Output means
300 . . . Patient-side terminal
400 . . . Blood pressure monitor
P . . . Patient
N . . . Communication network
Ar . . . Warning information
Tr . . . Measured value transition information
Me . . . Dosing information
An . . . Annotation information

1. A biometric information processing system, comprising:
a measured value acquisition device adapted to acquire a measured value related to biometric information of a patient;
a comparison device adapted to compare the measured value with an improvement target reference set in advance;
a dosing information acquisition device adapted to acquire dosing information including at least information on a dosing frequency of the patient when the measured value has not achieved the improvement target reference;
a medicinal effect confirmation support image generation device adapted to generate a medicinal effect confirmation support image in which measured value transition information indicates a change in the measured value of the patient at each first predetermined period and the dosing information are browsable when the measured value has not achieved the improvement target reference;
a warning information generation device adapted to generate warning information thereon when the measured value has not achieved the improvement target reference;
a first output device adapted to output the warning information when the measured value has not achieved the improvement target reference; and
a second output device adapted to output the medicinal effect confirmation support image when the measured value has not achieved the improvement target reference.

2. The biometric information processing system according to claim 1, wherein
the comparison device is adapted to compare an average value of the measured values in the first predetermined period with the improvement target reference; and
the dosing information acquisition device is adapted to acquire dosing information including information on a dosing frequency of the patient in the first predetermined period when the average value has not achieved the improvement target reference.

3. The biometric information processing system according to claim 1, wherein
the biometric information is information related to blood pressure;
the measured value acquisition device is adapted to acquire a systolic blood pressure value and a diastolic blood pressure value as the measured values for each measurement;
the improvement target reference to be compared by the comparison device is adapted to be set for each of the systolic blood pressure value and the diastolic blood pressure value;
the dosing information acquisition device is adapted to acquire the dosing information when either of the systolic blood pressure value or the diastolic blood pressure value has not achieved the improvement target reference set for each of the systolic blood pressure value and the diastolic blood pressure value; and
the warning information generation device is adapted to generate the warning information when either of the systolic blood pressure value or the diastolic blood pressure value has not achieved the improvement target reference set for each of the systolic blood pressure value and the diastolic blood pressure value.

4. The biometric information processing system according to claim 3, wherein
the measured value acquisition device is adapted to further acquire time period information for specifying a time period at which the blood pressure has been measured for each measurement; and
the measured value transition information indicates changes in the systolic blood pressure value and the diastolic blood pressure value at each of the first predetermined periods for each of the time periods.

5. The biometric information processing system according to claim 1, further comprising
a patient-side information processing terminal that executes, when the measured value has not achieved the improvement target reference, an automatic medical interview processing of requesting the patient to input at least information related to a dosing frequency of the patient, wherein
the dosing information acquisition device is adapted to acquire the dosing information input from the patient through automatic medical interview processing executed in the patient-side information processing terminal.

6. The biometric information processing system according to claim 5, wherein
the dosing information device is adapted to include information on a presence or absence and a type of a side effect caused in the patient by a medication; and
the automatic medical interview processing device is adapted to further request input of information on the presence or absence and the type of the side effect caused in the patient by the medication.

7. The biometric information processing system according to claim 5, further comprising
an action evaluation information generation device adapted to generate, based on the measured value and the dosing information in a second predetermined period, action evaluation information including information related to evaluation of an approach for treatment of the patient in the second predetermined period, wherein
the patient-side information processing terminal is adapted to output the action evaluation information.

8. The biometric information processing system according to claim 1, further comprising:
an action evaluation information generation device adapted to generate, based on the measured value and the dosing information in a second predetermined period, action evaluation information including information related to evaluation of an approach for treatment of the patient in the second predetermined period; and the patient-side information processing terminal to which the action evaluation information is output.

9. The biometric information processing system according to claim 1, further comprising
a recommended treatment information generation device adapted to generate, when the measured value has not achieved the improvement target reference, recommended treatment information related to a treatment strategy recommended to the patient, wherein
the recommended treatment information is further output to the second output.

10. The biometric information processing system according to claim 1, wherein
the medicinal effect confirmation support image includes information on a difference value between the measured value and the improvement target reference.

11. The biometric information processing system according to claim 1, further comprising
an annotation information generation device adapted to generate annotation information related to a matter to be noted by a medical professional in charge of the patient based on the measured value; wherein
the annotation information is further output to the second output.

12. The biometric information processing system according to claim 1, wherein
the improvement target reference is a value of the biometric information after a change that is assumed when the patient takes a medicine prescribed for the patient for the first predetermined period in accordance with a usage and a dose.

13. A biometric information processing device, comprising:
the measured value acquisition device;
the comparison device;
the dosing information acquisition device;
the medicinal effect confirmation support image generation device; and
the warning information generation device, wherein
the biometric information processing device constitutes at least a part of the biometric information processing system according to claim 1.

14. A biometric information processing method, comprising:
acquiring a measured value related to biometric information of a patient;
comparing the measured value with an improvement target reference set in advance;
acquiring dosing information including at least information on a dosing frequency of the patient when the measured value has not achieved the improvement target reference;
generating a medicinal effect confirmation support image in which measured value transition information indicating a change in the measured value of the patient at each first predetermined period and the dosing information are browsable when the measured value has not achieved the improvement target reference;
generating warning information thereon when the measured value has not achieved the improvement target reference;
outputting the warning information when the measured value has not achieved the improvement target reference; and
outputting the medicinal effect confirmation support image when the measured value has not achieved the improvement target reference.

15. A recording medium for causing a computer to function as a biometric information processing device according to claim 13.
